(19) United States
(12) Patent Application Publication
(10) Pub. No.: US 2022/0267730 A1
(43) Pub. Date: Aug. 25, 2022

(54) EX VIVO GENERATION OF IMMUNOCYTES RECOGNIZING BROTHER OF THE REGULATOR OF IMPRINTED SITES (BORIS) EXPRESSING CANCER STEM CELLS

(71) Applicant: Therapeutic Solutions International, Inc., Oceanside, CA (US)

(72) Inventors: Thomas E. Ichim, Oceanside, CA (US); Timothy G. Dixon, Oceanside, CA (US); Feng LIN, Oceanside, CA (US); Famela Ramos, Oceanside, CA (US); James Veltmeyer, Oceanside, CA (US)

(73) Assignee: Therapeutic Solutions International, Inc., Oceanside, CA (US)

(21) Appl. No.: 17/677,816

(22) Filed: Feb. 22, 2022

Related U.S. Application Data
(60) Provisional application No. 63/151,961, filed on Feb. 22, 2021.

(51) Int. Cl. C12N 5/0784; C12N 5/078

(52) U.S. Cl. CPC C12N 5/0639; C12N 5/0634; C12N 2501/22; C12N 2506/115; C12N 2502/11; C12N 2501/2304

(57) ABSTRACT

Disclosed are means, methods and compositions of matter useful for induction of immunity towards cancer stem cells by providing a dendritic cell, wherein said dendritic cells express BORIS and/or peptides derived from BORIS, wherein said dendritic cell is cultured in the presence of one or more immunocytes. In one embodiment said dendritic cells are derived from umbilical cord blood sources and allogeneic to T cells, which are expanded ex vivo and used for the purposes of immunotherapy.

### CROSS-REFERENCE TO RELATED APPLICATIONS

The present application claims the benefit of priority to U.S. Provisional Application No. 63/151,961, filed on Feb. 22, 2021, the contents of which are incorporated herein by reference.

### FIELD OF THE INVENTION

The invention pertains to the field of cancer immunotherapy, more specifically the invention pertains to the field of targeting cancer stem cells, more particularly the invention relates to generation ex vivo of T lymphocyte killer cells targeting cells expressing brother of the regulator of imprinted sites (BORIS) and/or derivatives of said BORIS.

### BACKGROUND

The utilization of the immune response for treatment of cancer offers the possibility of selectively targeting neoplastic tissue. Unfortunately, therapeutic reagents to tumor tissues has previously been attempted using immunological, metabolic, and molecular biology approaches, but with limited success. Among the major reasons for failure of such tumor-targeting therapies are the failure to identify tumor targets that are selective for the tumor versus non-tumor cells and essential for maintenance of tumor phenotype, the inability to inactivate targets, and the failure to kill cells expressing the target. Nevertheless, there remains an interest in developing selectively targeted therapies for cancer.

It is known that BORIS is an 11-zinc finger protein that is specifically expressed in neoplastically-transformed tissues, including tumor cell lines and primary patient samples, but is not expressed in non-transformed tissues with the exception of testis. The BORIS gene encodes a germ line, testis- and cancer-specific, paralog of the CTCF (CCCTC-binding factor; GenBank Accession No.: NM.sub.—006565), and is an epigentically-acting transcription factor that represses the tumor inhibitor functions of CTCF. Thus, BORIS is also referred to as CTCFL for CTCF-like. BORIS contains a central DNA-binding domain that is nearly identical to CTCF, but differs in N and C termini amino acid sequence, thereby suggesting that BORIS could play a role of interfering with CTCF-driven regulatory pathways if it is abnormally expressed in somatic cells. Abnormal activation of BORIS has been observed in all human primary tumors and cancer cell lines tested, including breast, lung, skin, bone, brain, colon, prostate, pancreas, mast cell, ovarian and uterine cancers, with increased expression associated with advanced stage of disease (see e.g., Ulaner et al., Hum. Mol. Genet. (2003)12:535-49; Vatolin et al., Cancer Res (2005) 65:7751-62; Hong et al., Cancer Res (2005) 65:7763-74; and Loukinov et al, J. Cell. Biochem. (2006) 98:1037-43; D'Arcy et al, Br. J. Cancer (2008) 98:571-9). BORIS induces de-repression of many genes associated with malignancy (Vatolin et al., Cancer Res (2005) 65:7751-62; Hong et al., Cancer Res (2005) 65:7763-74), and ectopic expression of BORIS in normal cells has been reported to result in classic features of cell-transformation (see Ghochikyan et al., J. Immunol. (2007) 178: 566-73).

Previous studies have demonstrated the potential of BORIS as a target for anti-cancer therapeutics. Protein-based, but not DNA-based, BORIS vaccine induced a significant level of antibody production in immunized animals, leading to breast cancer regression. Interestingly, potent anticancer CD8.sup.+-cytotoxic lymphocytes were generated after immunization with a DNA-based, but not protein-based, BORIS vaccine. (Ghochikyan et al., J. Immunol. (2007) 178: 566-73). However, the applicability of immunological approaches to cancer treatment is subject to limitations, including a) tumor suppression of the host immune system through active production of soluble and membrane bound factors; b) ability of tumor cells to lose expression of antigen processing machinery; and c) possibility of a deficit in the immunological repertoire of cancer patients caused by down regulation of TCR zeta chain expression.

### SUMMARY

Preferred embodiments include methods of inducing an immune response to cancer, said method comprising of: a) obtaining dendritic cell progenitors; b) culturing said dendritic cell progenitors in a manner to generate dendritic cells capable of antigen presentation; c) introducing to said dendritic cells one or more antigens found on cancer stem cells; d) culturing said dendritic cells with a population of immune cells; and e) providing conditions under which said immune cells can acquire ability to inhibit cell stem cells.

Preferred methods include embodiments wherein said dendritic cell progenitors are allogeneic to said immunocytes whose activation is desired.

Preferred methods include embodiments wherein said dendritic cell progenitors are autologous to said immunocytes whose activation is desired.

Preferred methods include embodiments wherein said dendritic cell progenitors are xenogeneic to said immunocytes whose activation is desired.

Preferred methods include embodiments wherein said dendritic cell progenitors are derived from monocytes.

Preferred methods include embodiments wherein said dendritic cell progenitors are derived from B cells.

Preferred methods include embodiments wherein said B cells express CD5.

Preferred methods include embodiments wherein said dendritic cell progenitors are derived from hematopoietic stem cells.

Preferred methods include embodiments wherein said hematopoietic stem cells are capable of generating leukocytic, lymphocytic, thrombocytic and erythrocytic cells when transplanted into an immunodeficient animal.

Preferred methods include embodiments wherein said hematopoietic stem cell expresses interleukin-3 receptor.

Preferred methods include embodiments wherein said hematopoietic stem cell expresses interleukin-1 receptor.

Preferred methods include embodiments wherein said hematopoietic stem cell expresses c-met.

Preferred methods include embodiments wherein said hematopoietic stem cell expresses mpl.

Preferred methods include embodiments wherein said hematopoietic stem cell expresses interleukin-11 receptor.

Preferred methods include embodiments wherein said hematopoietic stem cell expresses G-CSF receptor.

Preferred methods include embodiments wherein said hematopoietic stem cell expresses GM-CSF receptor.

Preferred methods include embodiments wherein said hematopoietic stem cell expresses M-CSF receptor.

Preferred methods include embodiments wherein said hematopoietic stem cell expresses VEGF-receptor.

Preferred methods include embodiments wherein said hematopoietic stem cell expresses c-kit.

Preferred methods include embodiments wherein said hematopoietic stem cell expresses CD33.

Preferred methods include embodiments wherein said hematopoietic stem cell expresses CD133.

Preferred methods include embodiments wherein said hematopoietic stem cell expresses CD34.

Preferred methods include embodiments wherein said hematopoietic stem cell expresses Fas ligand.

Preferred methods include embodiments wherein said hematopoietic stem cell does not express lineage markers.

Preferred methods include embodiments wherein said hematopoietic stem cell does not express CD14.

Preferred methods include embodiments wherein said hematopoietic stem cell does not express CD16.

Preferred methods include embodiments wherein said hematopoietic stem cell does not express CD3.

Preferred methods include embodiments wherein said hematopoietic stem cell does not express CD56.

Preferred methods include embodiments wherein said hematopoietic stem cell does not express CD38.

Preferred methods include embodiments wherein said hematopoietic stem cell does not express CD30.

Preferred methods include embodiments wherein said dendritic cells are generated by cultured in GM-CSF and interleukin-4 for a period of 2 days.

Preferred methods include embodiments wherein said dendritic cells are generated by cultured in GM-CSF and interleukin-4 for a period of 3 days.

Preferred methods include embodiments wherein said dendritic cells are generated by cultured in GM-CSF and interleukin-4 for a period of 4 days.

Preferred methods include embodiments wherein said dendritic cells are generated by cultured in GM-CSF and interleukin-4 for a period of 5 days.

Preferred methods include embodiments wherein said dendritic cells are generated by cultured in GM-CSF and interleukin-4 for a period of 6 days.

Preferred methods include embodiments wherein said dendritic cells are generated by cultured in GM-CSF and interleukin-4 for a period of 7 days.

Preferred methods include embodiments wherein said dendritic cell progenitors are further cultured in a concentration of lithium sufficient to inhibit GSK-3 more than 50%.

Preferred methods include embodiments wherein said lithium is a lithium salt.

Preferred methods include embodiments wherein said dendritic cell progenitors are further cultured in a concentration of an inhibitor of histone deacetylase an to inhibit HDAC activity more than 50%.

Preferred methods include embodiments wherein said dendritic cell progenitor is derived from a pluripotent stem cell.

Preferred methods include embodiments wherein said pluripotent stem cell is a parthenogenic derived cell.

Preferred methods include embodiments wherein said pluripotent stem cell is an inducible pluripotent stem cell.

Preferred methods include embodiments wherein said pluripotent stem cell is a somatic cell nuclear transfer derived stem cell.

Preferred methods include embodiments wherein said dendritic cell is activated with an agonist of an immune receptor.

Preferred methods include embodiments wherein said immune receptor is TLR-1.

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03

### BRIEF DESCRIPTION OF THE FIGURES

FIG. 1 is a bar graph showing treated cells predominantly killed CD133+ cancer cells

FIG. 2 is a bar graph showing treated cells predominantly killed PC-3 flk+ cancer cells

FIG. 3 is a bar graph showing treated cells predominantly killed PC-3 Rhodamine+ cancer cells

### DESCRIPTION OF THE INVENTION

The invention teaches the use of dendritic cells, especially allogeneic dendritic cells, as a means of stimulating generation of cytotoxic immune cells specific to BORIS. In one embodiment, BORIS is selected as a aa particularly appealing target for cancer therapy for several reasons. First, the widespread distribution of BORIS in different types of cancer cells coupled with the general concept that while non-malignant cells do not require activated oncogenes for survival, the suppression of an activated oncogene in a cancer cell often leads to apoptosis, suggests that therapies targeting BORIS may be effective for selective killing of a large number of cancer cell types. Thus, a single approach to cancer therapy may be applicable to many forms of cancer. Furthermore, BORIS is limited to testes and cancer cell types, and is not found in the vast majority of normal cell types. Therapies directed at BORIS are expected to have fewer side effects than others that target molecules or mechanisms present in normal cells, particularly in women where BORIS is not found in normal tissues. The physiological function of BORIS is reportedly related to erasure of methylation patterns during the process of spermatogenesis and hence the only expression of this gene in normal tissues is in the testis. BORIS is reportedly an epigenetic-acting oncogene that is thought to induce derepression of other oncogenes by inhibiting activity of the tumor suppressor gene, the CCCTC-binding factor (CTCF). CTCF was originally identified by its ability to suppress expression of the c-myc oncogene. Specifically, CTCF protein was reported to selectively bind CCCTC repeats in DNA upstream of the c-myc transcription start site. Deletion of CTCF binding regions was associated with upregulation of c-myc transcription. CTCF has also been reported to repress transcription of additional oncogenes including p27, p21, p53, p19 (ARF) and telomerase. The importance of CTCF as a tumor suppressor gene has been demonstrated by studies showing that mutation of CTCF results in oncogenesis and the finding that tumors express mutated CTCF. It has been speculated that BORIS selectively inactivates CTCF activity, thereby derepressing transcription of various oncogenes which ultimately results in the process of oncogenesis (4, 12).

“BORIS” or “the Brother of the Regulator of Imprinted Sites” protein, as used herein, refers to an epigenetically-acting zinc finger polypeptide present in mammalian testes and cancer cells, with an amino acid sequence that has greater than about 80% amino acid sequence identity, typically greater than 85% identity, often greater than 90% identity, and preferably 91%, 92%, 93%, 94%, 95%, 96%, 97%, 98% or 99% or greater amino acid sequence identity, to the BORIS amino acid sequence detailed in GenBank Accession No. AAM28645 (posted May 16, 2002). Implicitly encompassed by this definition are splice variants, variants containing conservative amino acid substitutions, and polymorphic variants capable of transforming a mammalian cell. The skilled artisan will be aware of methods for determining whether a polymorphic variant of BORIS is capable of transforming a mammalian cell, such as by transfection of a nucleic acid encoding the variant into a cell and e.g. observing colony formation. Typically, cancer cells that express BORIS have the amino acid sequence of GenBank Accession No. AAM28645, a splice variant thereof, a variant containing one or more conservative amino acid substitutions, or a polymorphic variant thereof that is capable of transforming a mammalian cell.

Identity is determined over a region of at least 20, 50, 100, 200, 500, or more contiguous amino acids. The terms “identical” or percent “identity,” as used herein in the context of two or more nucleic acids or amino acid sequences, refer to two or more sequences or subsequences that are the same or have a specified percentage of amino acid residues or nucleotides that are the same when compared and aligned for maximum correspondence over a comparison window (i.e. region). The definition includes sequences that have deletions, insertions and substitutions and may also be applied to the complement of a sequence (e.g. “100% complementary” polynucleotides). Preferably, identity is measured over the length of the polynucleotide or polypeptide, but is typically measured over a region that is at least about 20 amino acids or nucleotides in length, and often over a region that is at least 50-100 amino acids or nucleotides in length.

To calculate percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value, which is usually rounded to the nearest integer. It will be appreciated that the length of the aligned region can be a portion of one or both sequences up to the full-length of the shortest sequence. It will be appreciated that a single sequence can align differently with other sequences and hence, can have different percent sequence identity values over each aligned region.

The alignment of two or more sequences to determine percent sequence identity can be performed manually, by visual alignment, or can use computer programs that are well known in the art. For example, the algorithm described by Altschul et al. (1997, Nucleic Acids Res., 25:3389 402) can be used. This algorithm is incorporated into BLAST (basic local alignment search tool) programs, available at ncbi.nlm.nih.gov on the World Wide Web. BLAST searches can be performed to determine percent sequence identity between a nucleic acid molecule or polypeptide of the invention and any other sequence or portion thereof.

“BORIS gene” or “BORIS polynucleotide” refer to a polynucleotide sequence encoding a BORIS polypeptide, which is transcribed into an mRNA with at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the BORIS cDNA sequence of GenBank Accession No. AF336042 (posted May 16, 2002).

BORIS nucleic acid sequences also implicitly encompass “splice variants.” Similarly, BORIS polypeptides implicitly encompass any protein encoded by a splice variant of a BORIS nucleic acid. “Splice variant,” as used herein, refers to the products of alternative splicing of a gene. After transcription, an initial nucleic acid transcript may be spliced such that alternate nucleic acids are produced from the same template. Mechanisms for the production of splice variants include alternate splicing of exons. Alternate polypeptides derived from the same nucleic acid by read-through transcription are also encompassed by this definition. Any products of a splicing reaction, including recombinant forms of the splice products, are included in this definition.

“CTCF” as used herein refers to CCCTC-binding factor, a paralog of BORIS that is expressed in normal mammalian cells, and which typically has about 66% amino acid sequence identity to BORIS. “CTCF gene” refers to a polynucleotide sequence encoding a CTCF polypeptide, which is transcribed into an mRNA have a nucleotide sequence that has at least at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the CTCF cDNA sequence of GenBank Accession No.: NM.sub.—006565 (posted Jul. 20, 2008) or.

The terms “polynucleotide,” “nucleic acid,” and “nucleic acid molecule,” are used interchangeably herein to refer to polymeric forms of nucleotides of any length. The polynucleotides can contain deoxyribonucleotides, ribonucleotides, and/or their analogs. Polynucleotides can have any three-dimensional structure, and can perform any function, known or unknown. The term polynucleotide includes single-stranded, double-stranded, and triple helical molecules, and encompasses nucleic acids containing nucleotide analogs or modified backbone residues or linkages, which can be synthetic, naturally occurring, or non-naturally occurring, and which have similar binding properties as the reference nucleic acid. In particular, interfering RNAs (e.g., siRNA, shRNA) of the invention, can contain modifications or may incorporate analogs provided these do not interfere with the ability of the interfering RNA to inactivate homologous mRNA. Examples include replacement of one or more phosphodiester bonds with phosphorothioate linkages; modifications at the 2′-position of the pentose sugar in RNA, such as incorporation of 2-O-methyl ribonucleotides, 2′-H ribonucleotides, 2′-deoxy-2′-fluoro ribonucleotides (e.g. 2′-deoxy-2′-fluorouridine), or 2′-deoxy ribonucleotides; incorporation of universal base nucleotides, 5-C-methyl nucleotides, inverted deoxyabasic residues, or locked nucleic acid (LNA), which contains a methylene linkage between the 2′ and the 4′ position of the ribose.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

Exemplary embodiments of polynucleotides include, without limitation, genes, gene fragments, exons, introns, mRNA, tRNA, rRNA, interfering RNA, siRNA, shRNA, miRNA, anti-sense RNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers generally to polynucleotides that are between 5 and about 100 nucleotides of single- or double-stranded DNA. For the purposes of this disclosure, the lower limit of the size of an oligonucleotide is two, and there is no upper limit to the length of an oligonucleotide. Oligonucleotides are also known as “oligomers” or “oligos” and can be prepared by any method known in the art including isolation from naturally-occurring polynucleotides, enzymatic synthesis and chemical synthesis.

The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The terms “polypeptide,” “peptide” and “protein” are used interchangeably herein to refer to a polymer of amino acid residues of any length. Polypeptides can have any three-dimensional structure, and can perform any function, known or unknown. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid mimetics refers to chemical compounds that have a structure that is different from the general chemical structure of an amino acid, but that functions in a manner similar to a naturally occurring amino acid.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha..beta. T cell expressing TCR .alpha. .beta. chains, and a .gamma. .delta. T cell expressing TCR .gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used.

In one embodiment of the invention, BORIS and/or BORIS peptides are used to pulse dendritic cells. Said Dendritic cells are admixed with T cells. In one embodiment of the invention PBMCs are derived from leukapheresis and stimulated with anti-CD3 (OKT3, Ortho Biotech, Raritan, N.J.) and human recombinant IL-2 (600 IU/mL; Chiron, Emeryville, Calif.). After 3 days of culture, .about.5.times.107 to 1.times.108 lymphocytes are taken and transduced with retroviral vector supernatant (Cell Genesys, San Francisco, Calif.) encoding the chimeric CAR T recognizing tumor-endothelium specific antigen and subsequently selected for gene integration by culture in G418. In another embodiment the generation of dual-specific T cells is performed, stimulation of T cells is achieved by coculture of patient PBMCs with irradiated (5,000 cGy) allogeneic donor PBMCs from cryopre-served apheresis product (mixed lymphocyte reaction). The MHC haplotype of allogeneic donors is determined before use, and donors that differed in at least four MHC class I alleles from the patient are used. Culture medium consisted of AimV medium (Invitrogen, Carlsbad, Calif.) supplemented with 5% human AB-serum (Valley Biomedical, Winchester, Va.), penicillin (50 units/mL), streptomycin (50 mg/mL; Bio Whittaker, Walkersville, Md.), amphotericin B (Fungizone, 1.25 mg/mL; Biofluids, Rockville, Md.), L-glutamine (2 mmol/L; Mediatech, Herndon, Va.), and human recombinant IL-2 (Proleukin, 300 IU/mL; Chiron). Mixed lymphocyte reaction consisted of 2.times.106 patient PBMCs and 1.times.107 allogeneic stimulator PBMCs in 2 mL AimV per well in 24-well plates. Between 24 and 48 wells are cultured per patient for 3 days, at which time transduction is done by aspirating 1.5 mL of medium and replacing with 2.0 mL retroviral supernatant containing 300 IU/mL IL-2, 10 mmol/L HEPES, and 8.mu.g/mL polybrene (Sigma, St. Louis, Mo.) followed by covering with plastic wrap and centrifugation at 1,000.times.g for 1 hour at room temperature. After overnight culture at 37.degree. C./5% CO2, transduction is repeated on the following day, and then medium was replaced after another 24 hours. Cells are then resuspended at 1.times.106/mL in fresh medium containing 0.5 mg/mL G418 (Invitrogen) in 175-cm2 flasks for 5 days before resuspension in media lacking G418. Cells are expanded to 2.times.109 and then restimulated with allogeneic PBMCs from the same donor to enrich for T cells specific for the donor allogeneic haplotype. Restimulation is done by incubating patient T cells (1.times.106/mL) and stimulator PBMCs (2.times.106/mL) in 3-liter Fenwall culture bags in AimV+additives and IL-2 (no G418). Cell numbers were adjusted to 1.times.106/mL, and IL-2 was added every 2 days, until sufficient numbers for treatment were achieved.

In some embodiments of the invention adjuvants are added to T cells which have been expanded ex vivo against BORIS antigen. Said adjuvant may be selected from monophosphoryl Lipid A/synthetic trehalose dicorynomycolate (MPL-TDM), AS021/AS02, nonionic block co-polymer adjuvants, CRL 1005, aluminum phosphates, AIPO4), R-848, imiquimod, PAM3CYS, poly (I:C), loxoribine, bacille Calmette-Guerin (BCG), Corynebacterium parvum, CpG oligodeoxynucleotides (ODN), cholera toxin derived antigens, CTA 1-DD, lipopolysaccharide adjuvants, complete Freund's adjuvant, incomplete Freund's adjuvant, saponin, mineral gels, aluminum hydroxide, surface active substances, lysolecithin, pluronic polyols, polyanions, peptides, oil or hydrocarbon emulsions in water, MF59, Montanide ISA 720, keyhole limpet hemocyanins (KLH), dinitrophenol, and combinations thereof.

### EXAMPLES

### Stimulation of Immunity to CD133 Positive PC-3 Cancer Stem Cells

Cord blood generated allogeneic dendritic cells were obtained by culture of adherent monocytes with GM-CSF (100 IU/ml) and IL-4 (100 IU/ml) for 7 days. Cells expressed CD11c and possessed typical dendritic morphology. Cells where incubated with full length BORIS protein and treated with 30 ng/ml Poly (IC) for 24 hours to induce dendritic cell maturation. Dendritic cells where cultured with allogeneic peripheral blood mononuclear cells for 7 days in the presence of IL-2 (10 IU/ml) and anti-CD3 and anti-CD28 beads (10(5) beads per ml. T cells where purified by Magnetic Activated Cell Sorting for CD3 (containing both CD8 and CD4 cells). PC-3 prostate cancer cells where cultured and separated into either control (no T cells added), unfractionated, CD133 negative and CD133 positive. Cells where co-cultured at a 1:1 ratio and cytotoxicity was determined by MTT assay.

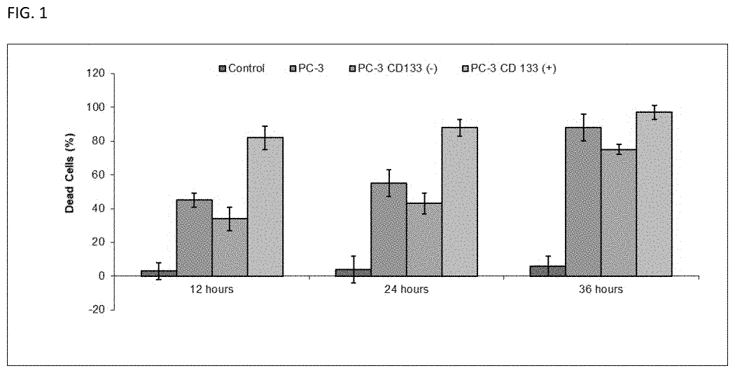

As observed, T cells possessed a preferential ability to kill CD133+ cells, which corresponds to a cancer stem cell phenotype. Results are shown in FIG. 1.

### Stimulation of Immunity to Fik Positive PC-3 Cancer Stem Cells

Cord blood generated allogeneic dendritic cells were obtained by culture of adherent monocytes with GM-CSF (100 IU/ml) and IL-4 (100 IU/ml) for 7 days. Cells expressed CD11c and possessed typical dendritic morphology. Cells where incubated with full length BORIS protein and treated with 30 ng/ml Poly (IC) for 24 hours to induce dendritic cell maturation. Dendritic cells where cultured with allogeneic peripheral blood mononuclear cells for 7 days in the presence of IL-2 (10 IU/ml) and anti-CD3 and anti-CD28 beads (10(5) beads per ml. T cells where purified by Magnetic Activated Cell Sorting for CD3 (containing both CD8 and CD4 cells). PC-3 prostate cancer cells where cultured and separated into either control (no T cells added), unfractionated, flk negative and flk positive. Cells where co-cultured at a 1:1 ratio and cytotoxicity was determined by MTT assay.

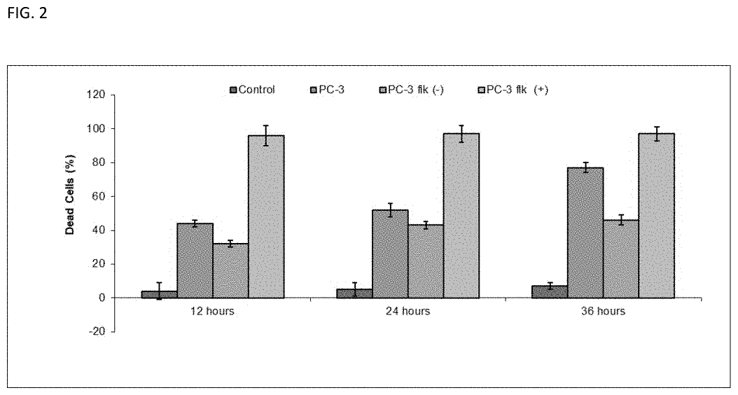

As observed, T cells possessed a preferential ability to kill PC-3 flk+ cells, which corresponds to a cancer stem cell phenotype. Results are shown in FIG. 2.

### Stimulation of Immunity to Rhodamine Positive PC-3 Cancer Stem Cells

Cord blood generated allogeneic dendritic cells were obtained by culture of adherent monocytes with GM-CSF (100 IU/ml) and IL-4 (100 IU/ml) for 7 days. Cells expressed CD11c and possessed typical dendritic morphology. Cells where incubated with full length BORIS protein and treated with 30 ng/ml Poly (IC) for 24 hours to induce dendritic cell maturation. Dendritic cells where cultured with allogeneic peripheral blood mononuclear cells for 7 days in the presence of IL-2 (10 IU/ml) and anti-CD3 and anti-CD28 beads (10(5) beads per ml. T cells where purified by Magnetic Activated Cell Sorting for CD3 (containing both CD8 and CD4 cells). PC-3 prostate cancer cells where cultured and separated into either control (no T cells added), unfractionated, Rhodamin negative and Rhodamin positive. Cells where co-cultured at a 1:1 ratio and cytotoxicity was determined by MTT assay.

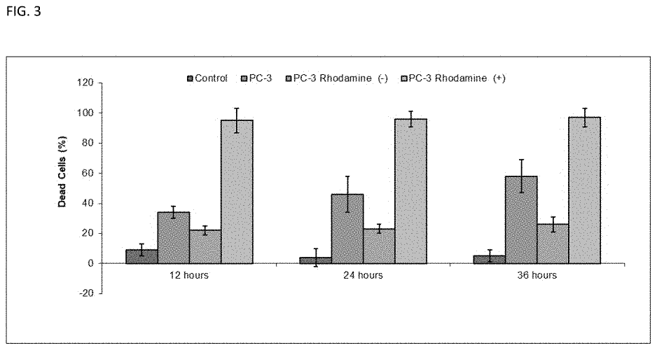

As observed, T cells possessed a preferential ability to kill PC-3 Rhodamin+ cells, which corresponds to a cancer stem cell phenotype. Results are shown in FIG. 3.

1. A method of inducing an immune response to cancer, said method comprising of: a) obtaining dendritic cell progenitors; b) culturing said dendritic cell progenitors in a manner to generate dendritic cells capable of antigen presentation; c) introducing to said dendritic cells one or more antigens found on cancer stem cells; d) culturing said dendritic cells with a population of immune cells; and e) providing conditions under which said immune cells can acquire ability to inhibit cell stem cells.

2. The method of claim 1, wherein said dendritic cell progenitors are allogeneic or autologous or xenogeneic to said immunocytes whose activation is desired.

3. The method of claim 1, wherein said dendritic cell progenitors are derived from monocytes.

4. The method of claim 1, wherein said dendritic cell progenitors are derived from B cells.

5. The method of claim 4, wherein said B cells express CD5.

6. The method of claim 1, wherein said dendritic cell progenitors are derived from hematopoietic stem cells.

7. The method of claim 6, wherein said hematopoietic stem cells are capable of generating leukocytic, lymphocytic, thrombocytic and erythrocytic cells when transplanted into an immunodeficient animal.

8. The method of claim 6, wherein said hematopoietic stem cell expresses interleukin-3 receptor.

9. The method of claim 6, wherein said hematopoietic stem cell expresses interleukin-1 receptor.

10. The method of claim 6, wherein said hematopoietic stem cell expresses c-met.

11. The method of claim 6, wherein said hematopoietic stem cell expresses mpl.

12. The method of claim 6, wherein said hematopoietic stem cell expresses interleukin-11 receptor.

13. The method of claim 6, wherein said hematopoietic stem cell expresses G-CSF receptor.

14. The method of claim 6, wherein said hematopoietic stem cell expresses GM-CSF receptor.

15. The method of claim 6, wherein said hematopoietic stem cell expresses M-CSF receptor.

16. The method of claim 6, wherein said hematopoietic stem cell expresses VEGF-receptor.

17. The method of claim 6, wherein said hematopoietic stem cell expresses c-kit.

18. The method of claim 6, wherein said hematopoietic stem cell expresses CD33.

19. The method of claim 6, wherein said hematopoietic stem cell expresses CD133.
